(19) United States
(12) Patent Application Publication
(10) Pub. No.: US 2026/0303972 A1
(43) Pub. Date: Oct. 1, 2026

(54) 3D SCANNER FOR MINIMIZING MOTION BLUR

(71) Applicant: 3SHAPE A/S, Copenhagen K (DK)

(72) Inventor: Karsten Bjerrum DIDERIKSEN, Copenhagen K (DK)

(73) Assignee: 3SHAPE A/S, Copenhagen K (DK)

(21) Appl. No.: 19/489,832

(22) PCT Filed: Jun. 6, 2024
(86) PCT No.: PCT/EP2024/065617

§ 371 Date: Dec. 3, 2025

(30) Foreign Application Priority Data
Jun. 7, 2023 (DK) PA202370273

(51) Int. Cl. H04N 23/68; A61B 1/045; A61B 1/05; A61B 1/06; A61B 1/24; A61C 9/00; H04N 23/73; H04N 23/74

(52) U.S. Cl. CPC H04N 23/689; A61B 1/045; A61B 1/051; A61B 1/0655; A61B 1/24; A61C 9/006; H04N 23/73; H04N 23/74

(57) ABSTRACT

An intraoral 3D scanner includes at least one projector unit including a light source configured for emitting light; and a pattern generating element configured for generating a pattern of light to be projected onto a surface of an object. The scanner further includes one or more camera units, operatively coupled to the projector unit, each camera unit including an image sensor for capturing one or more image frames, wherein the image sensor includes a two-dimensional array of pixels arranged in rows and columns and is configured for capturing a given image frame by sequentially exposing individual rows within the two-dimensional array of pixels, wherein each row is exposed for a predefined exposure time. The scanner is configured for sequentially switching the light source on and off at a predefined frequency, wherein the light source is turned on for a predefined duration during which all rows are exposed simultaneously.

### TECHNICAL FIELD

The present disclosure relates to an intraoral 3D scanner and associated 3D scanner system and method for capturing one or more image frames and generating a three-dimensional representation based hereof. In particular, the present disclosure relates to a 3D scanner for minimizing motion blur.

### BACKGROUND

3D scanning technology is widely used in dentistry for creating a digital impression of teeth. This technology enables dentists to obtain a detailed and accurate representation of the teeth in a digital format, which can then be used for a variety of applications, including the design and fabrication of dental restorations, orthodontic appliances, and other dental prostheses.

A rolling shutter sensor captures an image by exposing one row of pixels at a time, and then moving down the image sensor to capture the next row. This process continues until the entire image has been captured. Rolling shutter sensors are sometimes used in smartphones, tablets, and other consumer electronic devices.

However, the use of rolling shutter sensors presents several challenges. One of the primary challenges is the occurrence of distortion in the resulting images due to motion blur. The rolling shutter mechanism can cause distortion in the image, especially when there is movement in the scene being captured. This distortion can lead to inaccuracies in the resulting 3D model, which can have negative implications for the accuracy of dental restorations or orthodontic appliances.

Therefore, there is a need for improved methods and systems for using rolling shutter sensors in 3D scanning for generating digital impressions of teeth that overcome the challenge of motion blur, and provide a high degree of accuracy and precision.

### SUMMARY

Typically, optical 3D scanners and intraoral cameras utilize global shutter image sensors, since there are some inherent benefits of using global shutter over rolling shutter. One benefit is that all pixels on a global shutter image sensor are exposed simultaneously and read out simultaneously. Thus, a global shutter reads out the entire sensor at the same time when exposed, which means that there is no motion blur in the resulting image. However, global shutter sensors typically require more space, since they require more electronics in the form of transistors to operate; consequently leading to a larger package size. In intraoral 3D scanners it is advantageous to make the camera units very small, since it allows to package them closer together and consequently the resulting diameter or tip height of the 3D scanner can be made smaller. By utilizing a rolling shutter instead, the sensor is smaller for a given resolution, i.e. the pixel pitch is typically smaller for a rolling shutter sensor, whereby the individual camera units of the scanner can be made smaller.

However, using a rolling shutter in an intraoral 3D scanner introduces some challenges that need to be overcome. One main challenge is the presence of motion blur due to the relative motion between the 3D scanner and the object, and due to the individual image frames being exposed and/or read out sequentially, i.e. pixel row by pixel row. Thus, in rolling shutter sensors each pixel is typically exposed for the same amount of time, but all pixels are not exposed at the same time; i.e., they are not exposed globally. This type of operation risks introducing blur in the acquired images due to relative movement. Another challenge with using rolling shutter sensors for dental 3D scanning is the need for precise synchronization with other hardware components. Any deviations in timing can result in errors in the final 3D model, which can compromise the accuracy of the digital impression. Yet another challenge with rolling shutter sensors is to switch light sources without having to skip frames that have been exposed to two different light sources. This is due to the fact that light from two light sources will generally overlap the exposure time of a given frame when using rolling shutter, which implies that said frame has to be discarded if not for the flashing technique disclosed herein.

The presently disclosed 3D scanner addresses these challenges by utilizing a flashing light source, which is turned on for a predefined duration during which all rows on the sensor are exposed simultaneously, and preferably within the same readout frame. Thus, even though the individual rows are exposed in a sequential manner, there is a time window, where all pixels are exposed simultaneously and accumulate or generate charge carriers. By flashing the object with light during said time window, motion blur is avoided or minimized. A further advantage of the flash is that it avoids rolling shutter distortion. A further advantage of exposing the pixels simultaneously during the time window of the flash, is that it enables the use of two or more imaging modalities with different light sources, such that all image frames can be used, since there is no overlap in exposure of frames with different light sources.

Accordingly, the present disclosure solves the above-mentioned challenges by providing an intraoral 3D scanner comprising:
at least one projector unit comprising:
   a light source configured for emitting light;
   a pattern generating element configured for generating a pattern of light to be projected onto a surface of an object;
one or more camera units, operatively coupled to the projector unit, each camera unit comprising:
   an image sensor for capturing one or more image frames, wherein the image sensor comprises a two-dimensional array of pixels arranged in rows and columns, wherein the image sensor is configured for capturing a given image frame by sequentially exposing and/or reading out individual rows within the two-dimensional array of pixels, wherein each row is exposed for a predefined exposure time; and
one or more control units configured for sequentially switching the light source on and off at a predefined frequency, wherein the light source is turned on for a predefined duration during which all rows are exposed simultaneously.

The step of switching the light source on and off at a predefined frequency may also be referred to herein as flashing the light source. The predefined duration, during which the light source is turned on, may also be referred to as a flash. The inventors have realized that by configuring the projector unit or light source to flash in a sequence, wherein the flash occurs in a time window during which all pixels on the sensor are exposed, the operation of the rolling shutter sensor mimics that of a global shutter sensor; thus, having the benefit of minimizing or avoiding motion blur. In other words, the 3D scanner is preferably configured to provide a flash of light during a window of common exposure for all pixels. Furthermore, the advantage of having a small image sensor, i.e. with a small package size, is obtained by utilizing a rolling shutter sensor with a small pixel pitch. Thus, the presently disclosed camera units and mode of operating the projector unit offer a good compromise between the benefits of both rolling shutter and global shutter sensors.

The present disclose further relates to a 3D scanner system comprising: an intraoral 3D scanner as disclosed herein; and one or more processors operatively coupled to the intraoral 3D scanner, wherein the processor(s) are configured for generating a three-dimensional representation of an object based on the captured images. The intraoral 3D scanner may be based on any suitable scanning principle, such as focus scanning, triangulation, stereo vision, structure from motion, confocal scanning, or other scanning principles. The three-dimensional representation may be generated by triangulating features in the captured images in order to determine points in 3D space based on triangulation. A triangulation-based 3D scanner is further described in PCT/EP2022/086763 “Systems and methods for generating a digital representation of a 3D object” and PCT/EP2023/058521 “Intraoral 3D scanning device for projecting a high-density light pattern” by the same applicant, both of which are incorporated herein by reference in their entirety.

The present disclose further relates to a method of generating a three-dimensional (3D) representation of an object, the method comprising the steps of:
sequentially switching a light source on and off at a predefined frequency, wherein the light source is turned on for a predefined duration; and
capturing two or more image frames, using two or more image sensors, wherein each image frame is captured by sequentially exposing and/or reading out individual rows within a two-dimensional array of pixels on each image sensor, wherein each row is exposed for a predefined exposure time, wherein the predefined duration of the light source overlaps the exposure time, such that the object is illuminated in a time period during which all rows are exposed simultaneously; and
generating a three-dimensional representation of the object based on the captured image frames.

The present disclose further relates to a method of acquiring one or more image frames using an intraoral 3D scanner, the method comprising the steps of:
providing a trigger signal to one or more image sensors forming part of the intraoral 3D scanner, wherein the trigger signal is configured for initiating the exposure or readout of a given image frame;
sequentially exposing individual rows within a two-dimensional array of pixels on each image sensor, wherein each row is exposed for a predefined exposure time, wherein the exposure of a first row in the array is initiated upon receiving the trigger signal;
sequentially switching a light source on and off at a predefined frequency, wherein the light source is turned on for a predefined duration during which all rows in the array are exposed simultaneously; and
reading out a given image frame during a predefined frame readout time, during which the light source is off.

The present disclose further relates to an intraoral 3D scanner comprising:
at least one projector unit comprising:
a light source configured for emitting light;
a pattern generating element configured for generating a pattern of light to be projected onto a surface of an object;
one or more camera units, operatively coupled to the projector unit, each camera unit comprising:
an image sensor for capturing one or more image frames, wherein the image sensor comprises a two-dimensional array of pixels arranged in rows and columns, wherein the image sensor is configured for capturing a given image frame by sequentially exposing individual rows within the two-dimensional array of pixels, wherein each row is exposed for a predefined exposure time; and
one or more control units configured for sequentially switching the light source between a high light intensity mode and a reduced light intensity mode at a predefined frequency, wherein the light source is in the high light intensity mode for a predefined duration during which all rows are exposed simultaneously.

In the high light intensity mode the light source generates more light than in the reduced light intensity mode. The light source may be off during the reduced light intensity mode or it may be on a reduced light intensity. Reading out a given image frame may be done during a predefined frame readout time, during which the light source is in the reduced light intensity mode.

The trigger signal may be provided by the aforementioned one or more control units. In some embodiments, one of the image sensors is configured as a control unit, also referred to as a “master” image sensor. Thus, one of the image sensors may be configured for providing the trigger signal. In other embodiments, a separate device or integrated circuit is configured for providing the trigger signal to the image sensors. The method may further comprise the step of generating a three-dimensional representation of the object based on the image frames read out by the image sensors.

### BRIEF DESCRIPTION OF THE DRAWINGS

FIG. 1 shows an exemplary sequence of image frame acquisition. In this embodiment, the 3D scanner is configured for generating a flashing sequence of light.

FIG. 2 shows another exemplary sequence of image frame acquisition. This example is essentially similar to the embodiment of FIG. 1, with the exception that some of the white light flashes have been replaced by light with another wavelength (e.g. UV and/or IR).

FIG. 3a shows an exemplary sequence of image frame acquisition using a 3D scanner. This example is largely similar to the example shown in FIG. 2; however, in this example, a plurality of white light flashes are provided between different imaging modalities.

FIG. 3B shows the light intensities of the sequence of image frame acquisition using a 3D scanner shown in FIG. 3a.

FIG. 4a shows an exemplary sequence of image frame acquisition using a 3D scanner. This example is largely similar to the example shown in FIG. 3a.

FIG. 4b shows the light intensities of the sequence of image frame acquisition using a 3D scanner shown in FIG. 4a.

FIG. 5a shows an exemplary sequence of image frame acquisition using a 3D scanner.

FIG. 5b shows the light intensities of the sequence of image frame acquisition using a 3D scanner shown in FIG. 5a.

FIG. 6a shows an exemplary sequence of image frame acquisition using a 3D scanner.

FIG. 6b shows the light intensities of the sequence of image frame acquisition using a 3D scanner shown in FIG. 6a.

FIG. 7 shows an embodiment of a camera unit for a 3D scanner according to the present disclosure. In this embodiment, the camera unit comprises an image sensor and one or more lenses mounted in a lens mount.

FIG. 8 shows an exploded view of an embodied scan unit for a 3D scanner according to the present disclosure. In this embodiment, the scan unit comprises a projector unit a plurality of camera units, such as four camera units.

FIG. 9 shows an embodiment of a scan unit for a 3D scanner according to the present disclosure, wherein the projector-and camera units are assembled in the fixation unit.

FIG. 10 shows an embodiment of an intraoral 3D scanner according to the present disclosure. In this embodiment, the 3D scanner comprises two scan units (801, 802) arranged in series to provide an extended field of view of the 3D scanner.

FIG. 11 shows an embodiment of a 3D scanner system according to the present disclosure. The scanner system may comprise an intraoral 3D scanner for acquiring images and/or 3D frames inside the oral cavity of a subject.

FIG. 12 shows a schematic of some components of an intraoral 3D scanner according to an embodiment of the present disclosure.

### DETAILED DESCRIPTION

### 3D Scanner

The presently disclosed 3D scanner may be an intraoral 3D scanner for acquiring images within an intraoral cavity of a subject. The scanner may be a handheld scanner, i.e. a device configured for being held with a human hand. The scanner may employ any suitable scanning principle such as triangulation-based scanning, stereo vision, structure from motion, confocal scanning, or other scanning principles.

In some embodiments, the scanner employs a triangulation-based scanning principle. As an example, the scanner may comprise a projector unit and one or more camera units for determining points in 3D space based on triangulation. As another example, the scanner comprises a projector unit and two or more camera units, wherein the camera units are configured to image the scanned object from separate views, i.e. from different directions. In particular, the camera units may be configured to acquire a set of images, wherein a correspondence problem is solved within said set of images based on triangulation. The images within the set of images may be acquired by separate camera units of the scanner. The 3D scanner may be configured to generate a single 3D sub-scan based on the set of images obtained simultaneously by two or more camera units of the scanner.

The intraoral 3D scanner may comprise at least one projector unit for projecting a pattern onto the surface of an object, such as an object within the intraoral cavity of a subject. The intraoral 3D scanner may further comprise one or more camera units, such as two or more camera units, for capturing one or more images of the object. More details of the projector unit and camera units are provided in the following description.

### Projector Unit

A projector unit may be understood herein as a device configured for projecting light onto a surface. This may be achieved by providing a projector unit comprising a light source and a pattern generating element for structuring light emitted by the light source. The light source may be configured to generate unpolarized light, such as unpolarized white light. The projector unit may further comprise one or more collimation lenses for collimating light from the light source; said collimation lenses may be located between the light source and the pattern generating element. The projector unit may further comprise one or more focus lenses for focusing light emitted by the light source. The focus lenses may be located after the pattern generating element and configured for focusing the light at a given predefined working distance from the projector unit.

The projector unit may be selected from the group of: Digital Light Processing (DLP) projectors using a micro mirror array for generating a time varying pattern, or diffractive optical elements (DOE), or front-lit reflective mask projectors, or micro-LED projectors, or Liquid crystal on silicon (LCOS) projectors, or back-lit mask projectors, wherein a light source is placed behind a mask having a spatial pattern. As an example, the mask may be a chrome-on-glass mask. In general, the projector unit is preferably configured for projecting structured light or a pattern of light onto the surface of the object being scanned. The pattern may be dynamic, i.e. such that the pattern changes over time, or the pattern may be static in time, i.e. such that the pattern remains the same over time.

In preferred embodiments, the projector unit is configured for generating a sequence of flashing light, e.g. achieved by controlling a light source within the projector unit. As an example, the light source may be configured for emitting a flash of light of a certain predefined duration. Preferably, said duration is selected or defined such that the duration of the flash overlaps with the exposure of the pixels on the image sensor. Thus, the scanner or projector unit may be configured to generate a flash of light, wherein the flash has a predefined duration during which all rows are exposed simultaneously. An advantage hereof is that motion blur is minimized. In some embodiments, the duration of the flash is selected from about 0.1 ms to about 15 ms, such as from about 2 ms to about 10 ms. The 3D scanner may be configured for sequentially switching the light source of the projector unit on and off at a predefined frequency. As an example, the predefined frequency may be selected from about 25 Hz to about 40 Hz. The frequency may correspond to the 3D frame rate of the 3D scanner, i.e. the number of 3D frames generated per second, such that the 3D frame rate is from about 25 frames per second to about 40 frames per second. In some embodiments, the 3D scanner is configured for acquiring one or more frames using a different imaging modality, such as ultraviolet (UV) or infrared (IR). This may be achieved using one or more additional light sources, such as UV or IR light emitting diodes, located in the 3D scanner. Thus, the 3D frame rate may in some cases be lower than the overall sub-scan rate of the scanner, e.g. in case some of the image frames for 3D reconstruction are substituted for image frames acquired using UV or IR.

In some embodiments, the 3D scanner may run in the different imaging modality continuously, i.e. additional light source(s) may constantly be on, optionally at varying intensities. In such embodiments at least some of the pixels of the image sensor may be configured to discriminate the wavelength of the light used in the different imaging modality, such that these pixels will not detect the wavelength of the light used in the different imaging modality. This may be achieved by providing these pixels with a light filter configured for attenuating the wavelength of the light used in the different imaging modality. As an example, if the different imaging modality is an IR modality, the IR light source will be on continuously, optionally at varying intensities, and at least some of the pixels of the image sensor may be configured for discriminating against light of IR wavelengths, so that these pixels will not detect light at IR wavelengths. Light of IR wavelengths may instead be detected by a subset of the pixels of the image sensor, which are configured for detecting IR, or by a secondary image sensor dedicated to IR, or whichever light is used in the different imaging modality. When providing spectral filters like this or a secondary image sensor, it may further be possible to combine two or more imaging modalities in the same image frame, e.g. UV and IR combined or 3D and IR combined, as one subset of pixels/camera only sees the light from one imaging modality, while the other subset/camera only sees the light from the other imaging modality.

To avoid over exposure in the different imaging modality, the intraoral 3D scanner may be configured for shorter exposure times in the image frames where the different imaging modality is captures, e.g. the image frames for IR may be shorter than the image frames for white or visible light.

It is envisaged that rather than flashing the light source and keeping the additional light source continuously on during scanning, the light source may be kept continuously on, optionally at varying intensities, and the additional light source may be flashed. As an example, the white or visible light may be kept continuously on during scanning, while the IR light source may be flashed during image frames where IR images are captured. It is noted that if spectral filters are provided as mentioned above, they can be realized at a pixel level, i.e. providing filters in front of certain pixels or having pixels that are sensitive to the relevant wavelengths of light, or at a camera level, i.e. by providing a filter on one of the lens elements.

The projector unit may comprise a light source for generating white light. An advantage hereof is that white light enables the scanner to acquire data or information relating to the surface geometry and to the surface color simultaneously. Consequently, the same set of images can be used to provide both geometry of the object, e.g. in terms of 3D data/a 3D representation, and color of the object. Hence, there is no need for an alignment of data relating to the recorded surface geometry and data relating to the recorded surface color in order to generate a digital 3D representation of the object expressing both color and geometry of the object.

Alternatively, the projector unit may comprise multiple light sources such as LEDs individually producing light of different wavelengths (such as red, green, and blue) that may be combined to form light comprising different wavelengths or a range of wavelengths. Thus, the light produced by the light source(s) may be defined by a wavelength defining a specific color, or a range of different wavelengths defining a combination of colors such as white light. In some embodiments, the projector unit comprises a laser diode, such as a blue or green laser diode for generating blue or green light, respectively. An advantage hereof is that a more efficient projector unit can be realized, which enables a faster exposure compared to utilizing e.g. a white light diode. It is thus understood that while white light is used as an example in multiple of the embodiments disclosed herein, the invention is not limited to white light but also encompasses a light source configured for generating light comprising one or more wavelengths in the visible spectrum or a one or more distributions of wavelengths in the visible spectrum.

### Pattern Generating Element

In some embodiments, the 3D scanner or projector unit comprises a pattern generating element configured for generating a pattern of light. In the present disclosure, the terms structured light, patterned light, and pattern of light, are used interchangeably. The structured light can be implemented using a variety of techniques, including fringe projection, non-coded light, coded light, and sinusoidal patterns.

In some embodiments, the pattern generating element is configured for generating the pattern of light based on fringe projection. Thus, the projector unit may be configured for projecting a series of fringes onto the object being scanned, which can be straight, sinusoidal, or other shapes. The fringes deform on the surface of the object, and the deformation may be captured by one or more camera units to generate a 3D model.

In some embodiments, the pattern generating element is configured for generating the pattern of light based on coded light. Thus, the projector unit may be configured for projecting a pattern of binary codes onto the object being scanned, where each code represents a unique position in 3D space. The pattern may be captured by one or more camera units. The codes may subsequently be decoded to determine the object's shape.

In some embodiments, the pattern generating element is configured for generating the pattern of light based on phase-shifting. Thus, the projector unit may be configured for projecting a series of patterns with known phase shifts onto the object being scanned. The phase shifts may be used to calculate the distance between the object and the camera units, which can be used to generate a 3D model.

The pattern generating element may be a mask, such as a chrome-on-glass mask, having a spatial pattern for defining the pattern of light. Thus, in some embodiments, the pattern generating element is a mask configured for structuring light received by the light source of the projector unit. The mask may be a transparency mask or transmission mask. The mask may be located between the light source and the one or more focus lenses.

In other embodiments, the pattern generating element is configured to utilize diffraction and/or refraction to generate a light pattern. Thus, the pattern generating element may be a diffractive optical element (DOE) for generating the light pattern.

### Pattern of Light

The projector unit may be configured for projecting a pattern of light defined by a plurality of projector rays when a light source of the projector unit is turned on. The pattern of light may be generated using a pattern generating element, preferably located in the projector unit. The use of a pattern of light may lead to a correspondence problem, where a correspondence between points in the light pattern and points seen by the camera unit(s) viewing the pattern needs to be determined. In some embodiments, the correspondence problem is solved jointly for groups of projector rays emanating from the projector unit. A system and method for solving the correspondence problem in the context of 3D scanning is further described in PCT/EP2022/086763 “Systems and methods for generating a digital representation of a 3D object” by the same applicant, and further in PA 2023 70115 “System and method of solving the correspondence problem in 3D scanning systems” by the same applicant, both of which are incorporated herein by reference in their entirety.

The pattern of light may resemble a polygonal pattern comprising a plurality of polygons. The polygons may be selected from the group of: triangles, rectangles, squares, pentagons, hexagons, and/or combinations thereof. Other polygons can also be envisioned. In general, the polygons may be composed of faces, edges, and corners. In preferred embodiments, the polygons are repeated in the pattern in a predefined manner. As an example, the pattern may comprise a plurality of repeating units, wherein each repeating unit comprises a predefined number of polygons, wherein the repeating units are repeated throughout the pattern. The pattern of light may comprise a predefined arrangement comprising any of stripes, squares, dots, triangles, rectangles, and/or combinations thereof. In some embodiments, the pattern is non-coded, such that no part of the pattern is unique.

In some embodiments, the generated pattern of light is a polygonal pattern, such as a checkerboard pattern comprising a plurality of checkers. Similar to a common checkerboard, the checkers in the pattern may have alternating dark and bright areas corresponding to areas of low light intensity (dark) and areas of high(er) light intensity (bright). In some embodiments the pattern of light is a checkerboard pattern comprising alternating squares of different intensity in light. In other embodiments, the light pattern comprises a distribution of discrete unconnected spots of light.

### Camera Unit

A camera unit may be understood herein as a device for capturing an image of an object. The camera unit may comprise an image sensor and optionally one or more lenses for focusing light onto said image sensor.

The image sensor may be an active-pixel sensor (APS), such as a complementary metal-oxide-semiconductor (CMOS) sensor, or alternatively a charge-coupled device (CCD). A CCD sensor uses a series of capacitors to transfer charge across the sensor, allowing the sensor to capture and read out an image. A CMOS sensor generally uses a grid of pixels that are read out individually, allowing for faster readout speeds and lower power consumption compared to CCD sensors. The image sensor may be a global shutter sensor or a rolling shutter sensor. Alternatively, the sensor may be a time-of-flight (ToF) sensor configured to measure the time it takes for a pulse of light to travel to an object and back, allowing the sensor to generate a depth map of the object.

The image sensor may be based on back-side illumination or front-side illumination. The image sensor may comprise an array of pixels arranged in rows and columns. The array of pixels may be a two-dimensional array. The sensor may further comprise a microlens array arranged on top of the array of pixels, such that there is a microlens arranged on top of each pixel in the array. In the case of a front-side illumination sensor, the color filter array is typically arranged between the microlens array and a substrate of photodiodes, wherein the photodiodes are arranged at the back of the sensor. A back-illuminated sensor contains the same elements, but arranges the wiring behind the substrate of photodiodes. This generally improves the chance of an input photon being captured by the sensing layer, i.e. the photodiodes.

The image sensor may further comprise a color filter array. The color filter array may comprise a plurality of tiny color filters placed over the pixels of the image sensor, e.g., such that there is one color filter placed over each pixel. In general, the color filter may filter the light by wavelength range, such that separate filtered intensities include information about the color of the light incident on the sensor. The color filter array may be an RGB filter configured to provide information about the intensity of light in red, green, and blue wavelength regions. The image data captured by the image sensor may be converted to a full-color image (with intensities of all three primary colors represented at each pixel), e.g., by usage of a demosaicing algorithm. Some examples of suitable color filter arrays include: Bayer filter, RGBE filter, RYYB filter, CYYM filter, CYGM filter, RGBW Bayer, Quad Bayer, RYYB Quad Bayer, Nonacell, RCCC, and RCCB. Other suitable color filter arrays may be envisioned. When utilizing the image sensor data, color pixels may be combined to monochrome pixels of 2×2 color pixels for 3D depth reconstruction. In this case, the resolution of the 3D depth reconstruction is lower than the full resolution of the image sensor in each direction. When obtaining texture (color) images the full native resolution is preferably utilized (with color filtered pixels).

Each camera unit may comprise one or more focus lenses for focusing light onto the image sensor of the given camera unit. In some embodiments, each camera unit comprises two or more lenses, or lens elements, assembled in a camera lens stack. Thus, each camera unit may comprise a camera lens stack comprising a plurality of lens elements. The purpose of the focus lens(es) or camera lens stack may be to define or ensure a predetermined focus distance, or working distance, of the camera unit. The camera lens stack may further define the camera optical axis. In some embodiments, the lens elements of the camera lens stack are attached together to form a single unit. In some embodiments, each camera unit has a field of view of 50-115 degrees, such as 65-100 degrees, such as 65-75 degrees. In other embodiments, each camera unit has a field of view of 80-90 degrees. The field of view of the camera units may be overlapping such that the camera units view the same scene or object, or they may be at least partly overlapping.

Each camera unit may further comprise a lens mount configured for receiving and mounting the focus lenses or camera lens stack. The lens mount may comprise a cylindrically shaped section adapted to receive said lenses/lens stack, such that said lens stack can be fixedly mounted herein. The lens mount may further comprise a flange adapted to interface with a fixation unit to ensure correct placement of the lens mount within the fixation unit in at least one direction. In some embodiments, the flange comprises one or more flat surfaces for interfacing with the fixation unit to fix the position of a given lens mount in the fixation unit in at least two directions. In some embodiments, the camera units are symmetrically arranged around the projector unit.

In some embodiments, the scanner comprises two or more camera units configured for acquiring a set of images comprising at least one image from each camera unit, wherein each image includes at least a portion of the projected pattern of light. In some embodiments, the images within the set of images are acquired simultaneously, e.g., by synchronizing the camera units using a trigger signal. Furthermore, the number of images in the set of images may correspond to the number of camera units, wherein each camera unit contributes one image to the set of images. An advantage hereof, is that the light-budget is improved; thus, less power is consumed by the light source and the projector unit. Consequently, less heat is generated by said components, which is desired, since oftentimes it is difficult to remove heat from intraoral scanners.

### Acquisition of Images

The image sensor of each camera unit may be configured for capturing a given image frame by sequentially exposing individual rows within the array of pixels, wherein each row is exposed for a predefined exposure time. The exposure time may be from about 10 ms to about 35 ms, such as from about 15 ms to about 30 ms, such as from about 18 ms to about 26 ms. In preferred embodiments, the exposure time is from about 18 ms to about 26 ms. The exposure time may correspond to the frame readout time plus the duration of a given flash, during which the light source is turned on. Accordingly, the exposure time is preferably larger than the frame readout time and/or the duration of the flash, even more preferably it corresponds approximately to the sum of the two. The image sensor may be configured to provide a global reset option where the exposure of all pixels starts synchronously. In that case, the read out of the pixel rows may still be performed sequentially.

The frame readout time corresponds to a time during which pixels on the image sensor are read out. Preferably, the entire array of pixels, i.e. all rows, are read out during the frame readout time, such that a given image frame is formed by the end of said readout time. Thus, the frame readout time may be understood as the time it takes for the image sensor to read out all the rows of the image after the exposure is complete. The frame readout time may be from about 5 ms to about 25 ms, such as from about 8 ms to about 20 ms, such as from about 12 ms to about 18 ms. Once the exposure time for the top row is over, the sensor is preferably configured to begin reading out the rows sequentially, starting from the top row and moving downwards.

The term “reading out” the rows or pixels from the image sensor may be understood as retrieving the stored electrical charge or voltage values from each individual pixel or row of pixels in order to convert them into digital data that represents the captured image. During the exposure time, each pixel in the sensor may accumulate photons and convert them into an electrical charge or voltage proportional to the incident light intensity. This charge or voltage typically represents the brightness level of that pixel in the captured image. After the exposure time, the charges or voltages from each pixel may be sequentially transferred to neighboring storage elements or amplifiers within the sensor. The transferred charges or voltages may then be amplified to appropriate levels to improve signal quality and reduce noise. Additionally, in some cases, the analog signals may undergo analog-to-digital conversion within the sensor itself to directly generate digital data. Once the charges or voltages have been amplified and converted, the sensor may proceed to read out the values row by row or pixel by pixel, typically starting from one row or corner of the sensor. This may involve sequentially accessing each storage element or amplifier to retrieve the corresponding charge or voltage value. The readout analog signals may then be converted into digital data using analog-to-digital converters (ADCs). The digital values may represent the brightness and/or color information of each pixel and may be further processed and/or stored for subsequent image processing or display. Thus, a given image may be output from the image sensor as either an analog or a digital signal.

The exposure of a given image frame may be initiated by a trigger signal. Preferably, a given image frame is exposed sequentially, such as row by row, beginning with the top row in the array of pixels. Thus the intraoral 3D scanner may be configured for providing a trigger signal to the one or more image sensors forming part of the scanner, wherein the trigger signal is configured for initiating the exposure or read out of a given image frame. Each image sensor may comprise a pin for receiving the trigger signal.

The trigger signal may be provided by one or more control units. In some embodiments, the control unit is a device selected from the group of: field-programmable gate array (FPGA), Digital Signal Processor (DSP), Application-Specific Integrated Circuit (ASIC), Microcontroller (MCU), Programmable System-on-Chip (PSoC), or System-on-Chip (SoC). Other devices capable of providing a trigger signal to the image sensors can be envisioned and utilized. In the case of multiple image sensors forming part of the 3D scanner, one of said sensors may be configured to provide the trigger signal to the remaining image sensors.

In some embodiments, one of the image sensors is configured as a control unit, also referred to as a “master” image sensor. Thus, one of the image sensors may be configured for providing the trigger signal. Thus, in some embodiments, a first image sensor comprised in a first camera unit is configured as the control unit, such that the first image sensor is configured for providing, to each of the remaining image sensors, a trigger signal for initiating the capture of a given image frame. In other embodiments, a separate control unit, such as an integrated circuit, is configured for providing the trigger signal to the image sensors. The 3D scanner may further be configured for generating a three-dimensional representation of the object based on the image frames read out by the image sensors.

### Multiple Camera Units

In some embodiments, the 3D scanner comprises a projector unit and a plurality of camera units, such as two or more camera units, or four or more camera units. Each camera unit comprises an image sensor, which may be configured as disclosed herein. The camera units may be synchronized such that they capture images approximately simultaneously. Said synchronization may be achieved via the aforementioned trigger signal, provided by e.g. a control unit or a “master” image sensor. Thus, in some embodiments each image sensor receives a trigger signal for each image frame capture, such that they are kept in close synchronization. The camera units may be configured for capturing a set of images, wherein each camera unit contributes at least one image to the set of images. In some embodiments, the number of images in the set of images corresponds to the number of camera units. An advantage hereof, is that the light-budget is improved; thus, less power is consumed by the light source and the projector unit. Consequently, less heat is generated by said components, which is desired, since oftentimes it is difficult to remove heat from intraoral scanners. As an example, in the case of four camera units, the set of images may contain four images. The camera units may be arranged to have at least partly overlapping fields of view along different camera optical axes. The camera units may view approximately the same scene or object.

### Processors

The 3D scanner system may comprise one or more processors configured to generate a 3D representation based on the set of images captured by the camera units. This may be achieved by identifying image features in the set of images and determining points in 3D space based on triangulation. In some embodiments, the location of the image features are identified using machine learning, such as a neural network. The one or more processors may include a neural processing unit (NPU) configured for executing one or more machine learning algorithms, such as for running the neural network for identifying image features. The processors may be located on the scanner or they may be located on an external computer. The 3D representation may be generated continuously during a scanning session, and/or it may be generated in real-time. The scanner system may further comprise a display for displaying the 3D representation. The rendering of the 3D representation and the display of said representation may further occur in real-time, or perceived real-time to the user, e.g. with time lags below 50 ms. The one or more processors may be selected from the group of: central processing units (CPU), accelerators (offload engines), general-purpose microprocessors, graphics processing units (GPU), neural processing units (NPU), application-specific integrated circuits (ASIC), field-programmable gate arrays (FPGA), dedicated logic circuitry, dedicated artificial intelligence processor units, or combinations thereof.

### DETAILED DESCRIPTION OF THE DRAWINGS

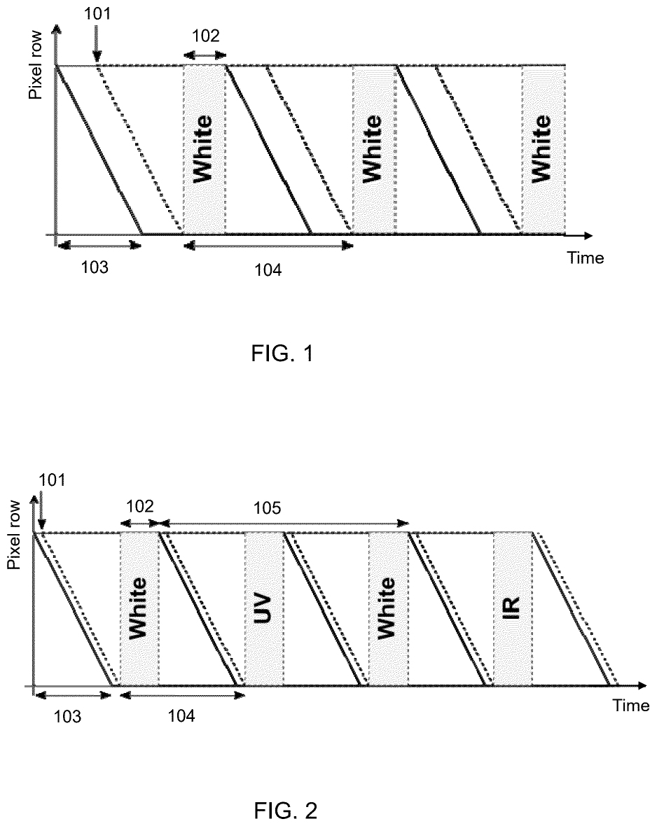

FIG. 1 shows an exemplary sequence of image frame acquisition using a 3D scanner. In this embodiment, the 3D scanner is configured for generating a flashing sequence of light of a predefined duration 102 during which all pixels on the image sensor(s) are exposed. Thus, the columns labelled “White” corresponds to a flash of white light of a certain duration 102, wherein the flash of light occurs during a window of common exposure for all pixels on the sensor(s). An advantage hereof is that motion blur is limited due to a relatively short and intense flash and that rolling shutter distortion is limited or entirely eliminated. The pulses labelled “White” are used to refer to the acquisition of 3D frames, not necessarily using white light. The white light is used as an example, and other known modalities for generating a 3D frame may be utilized. However, white light is advantageous since it allows the simultaneous capture of both 3D information and texture.

The 3D scanner may be configured for immediately initiating the readout 103 of a given image frame once the flash has occurred, i.e. when the light source is switched off again. In other words, the 3D scanner is preferably configured for initiating the readout of a given frame from the top row in the pixel array, once the exposure time 104 of said row has lapsed. After readout of the top row, and while reading out the remaining rows of a given image frame associated with the previous “pulse” or flash of light, the image sensor may be configured to initiate the exposure 101 of the next image frame, e.g. beginning from the top row again. Thus, the frame readout time 103 and the exposure 104 of at least some pixel rows may overlap. Some of the frames acquired using white light may be substituted for frames acquired using other imaging modalities, such as other wavelengths of light, e.g. UV or IR.

FIG. 2 shows another exemplary sequence of image frame acquisition using a 3D scanner. This example is essentially similar to the embodiment of FIG. 1, with the exception that some of the white light flashes 102 have been replaced by light with another wavelength; here UV and IR. In that case, the separation 105 of 3D frames is a bit increased, leading to a lower 3D frame rate. However, using the scanner in another imaging modality (UV, IR, etc.) allows for capturing data relevant for diagnostic purposes, such as for imaging and visualizing internal regions of the teeth and/or cariogenic regions, i.e. areas on the teeth with caries. Thus, the scanner may be configured for providing an excitation signal and receiving a corresponding fluorescence signal from the object, in this example labelled “UV”, wherein the excitation signal is suitable for exciting bacteria in the mouth of the subject, whereby cariogenic regions can be visualized based on the reflected fluorescence signal. As an example, the 3D scanner may be configured to emit blue light or ultraviolet light having a wavelength or range of wavelengths selected from the range of about 315 nm to about 425 nm. This may be achieved using one or more additional light sources placed in the 3D scanner, such as in the tip of said scanner. The infrared (IR) modality may similarly be used to determine areas or volumes of caries. In particular, some infrared wavelengths penetrate dental tissue, whereby volumes inside the dentin and enamel can be imaged and visualized. As an example, the 3D scanner may be configured to emit infrared light having a wavelength or range of wavelengths selected from the range of about 700 nm to about 1.5 μm. In some embodiments, the 3D scanner is configured for acquiring 3D data and texture using two different imaging modalities, e.g. provided by separate flashes. As an example, the scanner may be configured for acquiring 3D frames using a monochromatic pattern and white illumination for acquiring texture.

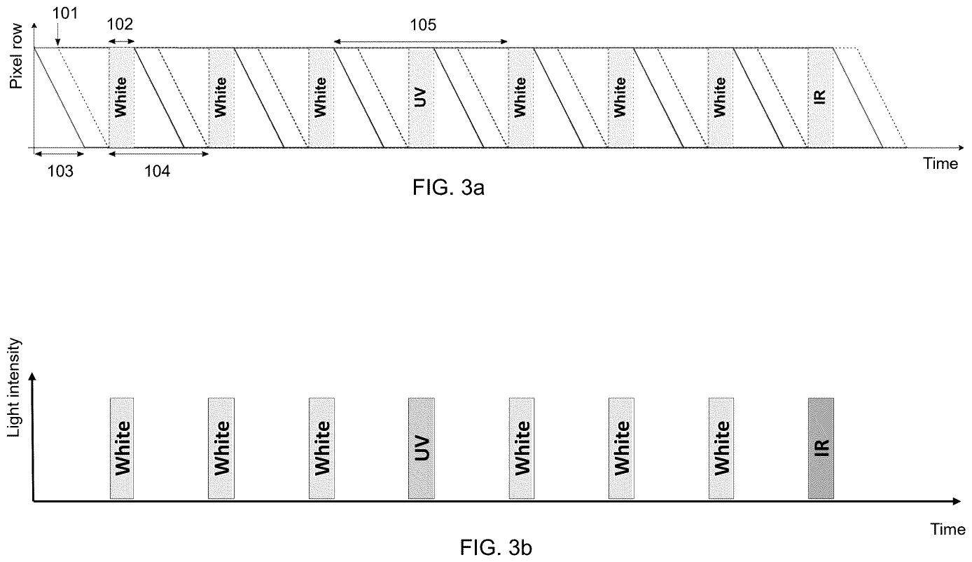

FIG. 3a shows an exemplary sequence of image frame acquisition using a 3D scanner. This example is largely similar to the example shown in FIG. 2; however, in this example, a plurality of white light flashes are provided between different imaging modalities (UV, IR, etc.). Thus, the 3D scanner may be configured for acquiring a plurality of 3D frames in succession, such as two or more 3D frames, or three or more 3D frames, before switching to another imaging modality. The 3D frames may also be acquired using another imaging modality than white light. Examples include fringe patterns, line patterns, dot patterns, monochromatic patterns, color patterns, static patterns, dynamic patterns, etc. Thus, the pulses labelled “White” are used to refer to the acquisition of 3D frames, and not necessarily using white light. The white light is used as an example, and other known modalities for generating a 3D frame may be utilized.

FIG. 3b shows the intensity of the light emitted by the light sources, i.e. the light source and the additional light sources, in the sequence of image frame acquisition using a 3D scanner depicted in FIG. 3a. As can be seen, the light source or additional light source is flashed during the image frames where image data corresponding to the respective imaging modality is captured, i.e. the white or visible light source is flashed during the acquisition of 3D frames, the UV light source is flashed during the acquisition of fluorescent images, and the IR light source is flashed during the acquisition of IR images. It is noted that while the light intensity is depicted as square pulses, this may be idealized, and each light source may experience a ramp-up and ramp-down time resulting in a trapezoidal pulses. It is further noted that the flashes themselves may be produces by a series of sub-flashes, as would be the case if the pulses are generated by pulse width modulation of the light source.

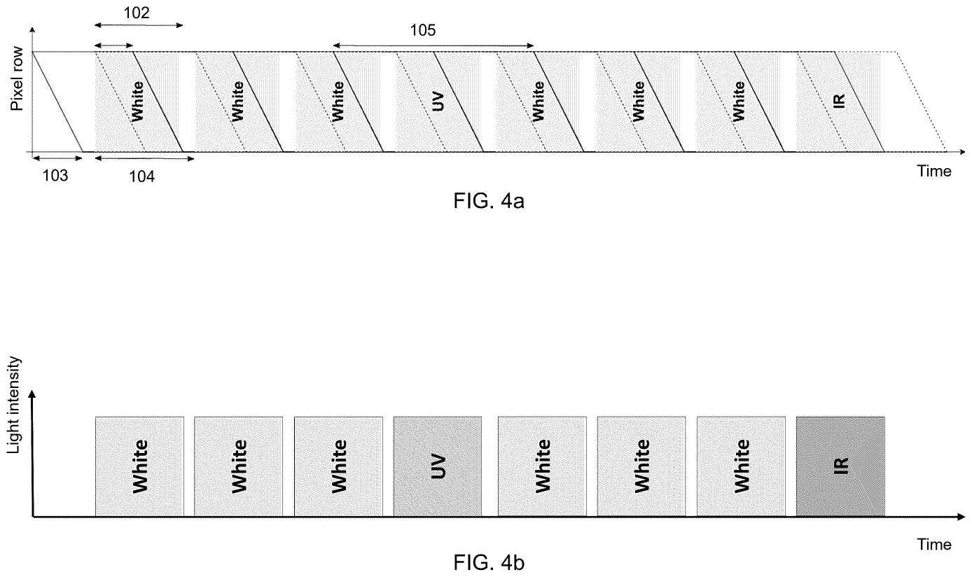

FIG. 4a shows an exemplary sequence of image frame acquisition using a 3D scanner. This example is largely similar to the example shown in FIG. 3a; however, in this example, a different light source is used for the acquisition of 3D frames. In this example, the light source is less bright and consumes less power; in this case, the light source is turned on for a longer time period 102. This embodiment has the advantage of saving power for the projector unit of the scanner.

FIG. 4b shows the intensity of the light emitted by the light sources, i.e. the light source and the additional light sources, in the sequence of image frame acquisition using a 3D scanner depicted in FIG. 4a. As can be seen, the light source or additional light source is flashed during the image frames where image data corresponding to the respective imaging modality is captured, i.e. the white or visible light source is flashed during the acquisition of 3D frames, the UV light source is flashed during the acquisition of fluorescent images, and the IR light source is flashed during the acquisition of IR images.

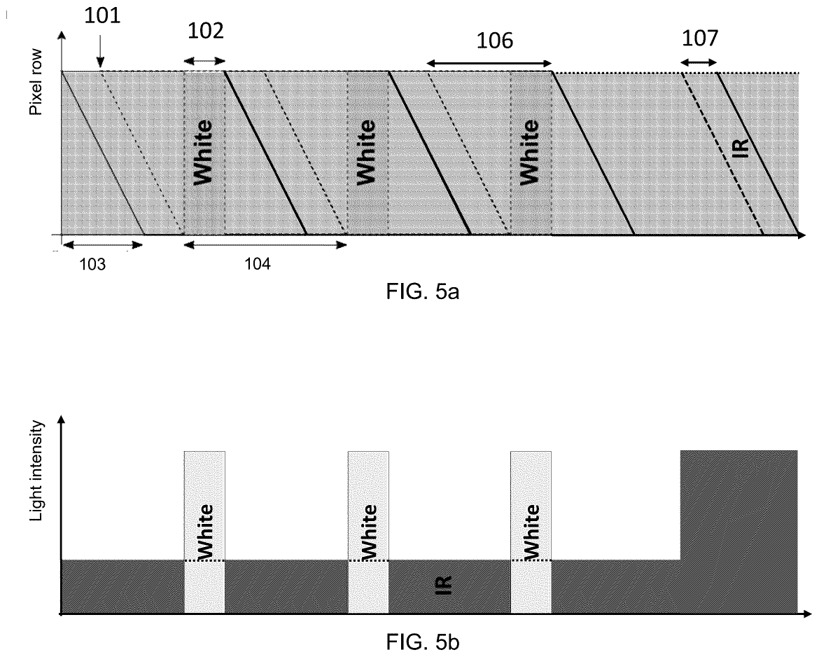

FIG. 5a shows an exemplary sequence of image frame acquisition using a 3D scanner. In this example, a light source used for the acquisition of 3D frames, i.e. white or visible light, is flashed periodically, except in the image frame where different imaging modalities are being acquired. In the shown example, every fourth image frame is used for acquisition of IR images, and thus every fourth flash of the white or visible light source is omitted. Because the IR images are not used for generating the 3D representation of the scanned object, motion blur is less of an issue in this imaging modality. Consequently, the IR light source need not be flashed, but can instead be kept continuously on during scanning, whereby the effect of ramp-up time as well as pulse transients resulting from the sudden increase in voltage that happens when a light source is flashed are reduced. This advantage may however also be achieved by maintaining the IR light source on at reduced intensity, i.e. non-zero, whereby power consumption and heat production of the IR light source may be reduced. Further, while the scanner emits light while inside the mouth of a patient, the user, e.g. the dentist, may still see parts of the emitted light. It is therefore a further advantage that the visible light is kept continuously on, as this might be perceived more pleasant for the user to see than periodical light flashes.

FIG. 5b shows the intensity of the light emitted by the light sources, i.e. the light source and the additional light sources, in the sequence of image frame acquisition using a 3D scanner depicted in FIG. 5a. As can be seen, the light source is flashed during the image frames where 3D data is acquired, i.e. the white or visible light source is flashed during the acquisition of 3D frames. Rather than flashing the IR and during the image frames reserved for the IR imaging modality, the IR light source is kept continuously on in a reduced light intensity mode during scanning, and switched to a high light intensity mode during the image frames where IR images are acquired.

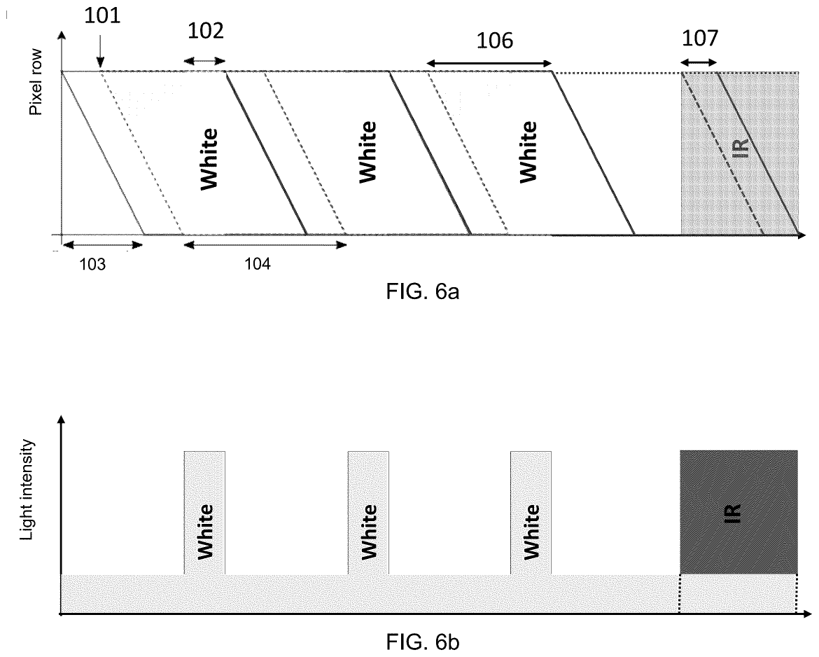

FIG. 6a shows an exemplary sequence of image frame acquisition using a 3D scanner. In this example, a light source used for the acquisition of 3D frames, i.e. white or visible light, is kept continuously on, albeit at reduced light intensity mode, and periodically switched to a high light intensity mode for a predefined duration during which all rows are exposed simultaneously. The period of high intensity light is skipped in the image frame where different imaging modalities are being acquired, e.g. IR or UV. In the shown example, every fourth image frame is used for acquisition of IR images, and during this imaging modality the IR light source is flashed.

By keeping the light source on in a reduced light intensity mode the effect of ramp-up time as well as pulse transients resulting from the sudden increase in voltage that happens when a light source is flashed are reduced. Because the different imaging modalities occupy fewer image frames than the image frames for acquisition of 3D data, it may be advantageous to suffer the consequences of flashing the light sources for these imaging modalities rather than keeping them on at reduced intensity to reduce power consumption.

FIG. 6b shows the intensity of the light emitted by the light sources, i.e. the light source and the additional light sources, in the sequence of image frame acquisition using a 3D scanner depicted in FIG. 6a. As can be seen, the light source is kept on in a reduced light intensity mode, except during the image frames where 3D data is acquired where the light source is switched to a high light intensity mode for a predefined duration during which all rows are exposed simultaneously. Keeping the light source on while scanning rather than flashing it reduces the effects of ramp-up and pulse transients. By confining the duration of the high intensity light mode to the period in which all rows are exposed simultaneously, motion blur from the rolling shutter can be limited to that caused by exposure of the pixels during the reduced light intensity mode, which due to the reduced exposure is less significant than if the light source was constantly the in high intensity mode.

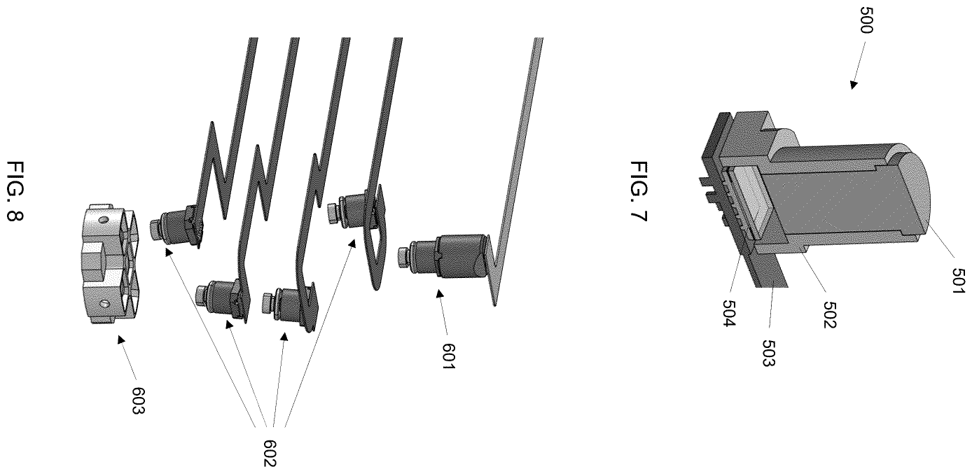

FIG. 7 shows an embodiment of a camera unit 500 for a 3D scanner according to the present disclosure. In this embodiment, the camera unit comprises an image sensor 504 connected to a flexible printed circuit board (PCB) 503, wherein the camera unit further comprises one or more lenses 501 mounted in a lens mount 502. The lenses 501 may be one or more focus lenses configured for focusing light received from the scanned object and onto the image sensor 504. The 3D scanner may comprise a plurality of such camera units 500.

FIG. 8 shows an exploded view of an embodied scan unit for a 3D scanner according to the present disclosure. In this embodiment, the scan unit comprises a projector unit 601 comprising a light source, and a plurality of camera units 602, such as four camera units 602. In this example, the scan unit further comprises a fixation unit 603 for receiving and mounting the projector unit 601 and the camera units 602, such that said unit may be fixedly mounted in the fixation unit 603. In some embodiments, the fixation unit 603 is made in a rigid material such as stainless steel. It may be made in one piece to provide a rigid structure for fixing the units.

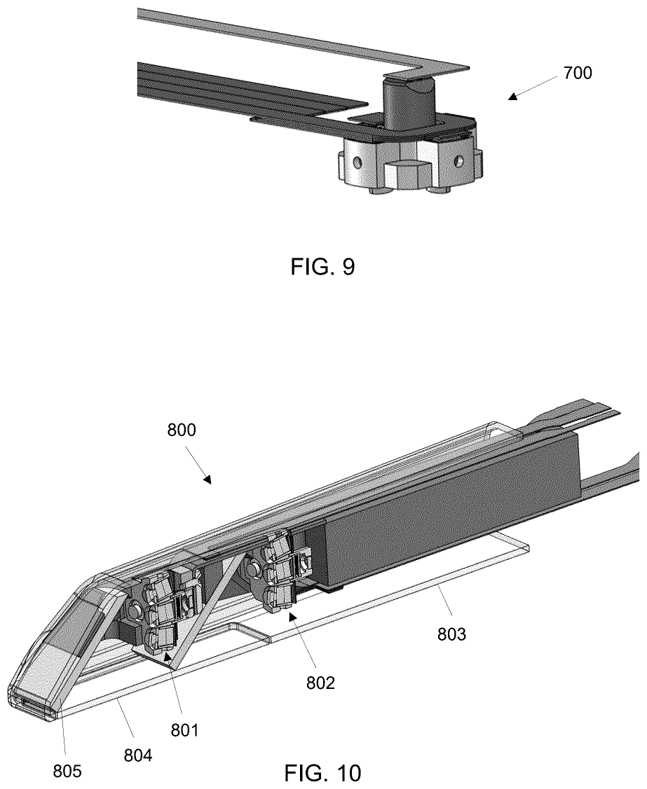

FIG. 9 shows an embodiment of a scan unit 700 for a 3D scanner according to the present disclosure. This embodiment is similar to the one shown in FIG. 6; however, in this figure the scan unit is shown when the projector-and camera units are assembled in the fixation unit. Each module or unit may be connected to a main printed circuit board via a flexible printed circuit board (PCB).

FIG. 10 shows an embodiment of an intraoral 3D scanner 800 according to the present disclosure. In this embodiment, the 3D scanner 800 comprises two scan units (801, 802) arranged in series to provide an extended field of view of the 3D scanner. However, other embodiments may only include a single scan unit, e.g. arranged in combination with a mirror 805. In this example, each scan unit (801, 802) is positioned behind a mirror 805, such that light projected from the projector unit is incident on the mirror 805, and reflected onto a surface of the scanned object. In other words, the direction of the light being projected is changed from a forward direction substantially parallel to the longitudinal axis of the scanner to a direction substantially orthogonal to said axis. These directions may apply only to a center beam of the projected pattern. Thus, other beams being projected may form a cone of light onto the mirror and/or surface of the object. Similarly, light reflected by the scanned object may be reflected by the mirrors 805 and onto the image sensors of the scan units (801, 802). An advantage of utilizing one or more mirrors 805 arranged in front of the scan units, is that the probe or tip height of the 3D scanner can be made smaller for a given field of view of the scanner, since a part of the optical path of the projector-and camera units can be ‘folded’ inside the scanner.

The 3D scanner may comprise a housing 803, and there may be provided one or more optical windows 804 flush with the surface of said housing 803. The housing 803 of the scanner 800 may form an elongated probe for being inserted into the oral cavity of a subject, such as a person. The housing may be provided in one piece, e.g. having one or more openings, preferably having optical windows arranged in said opening(s). The material of the optical window(s) may be Poly(methyl methacrylate) (PMMA) or glass, such as sapphire glass. A 3D scanner comprising two scan units arranged in series is further described in PCT/EP2023/058980 “Intraoral scanning device with extended field of view” by the same applicant, which is incorporated herein by reference in its entirety.

FIG. 11 shows an embodiment of a 3D scanner system 900 according to the present disclosure. The scanner system may comprise an intraoral 3D scanner 902 for acquiring images and/or 3D frames inside the oral cavity of a subject. In particular, the 3D scanner may be configured for acquiring images and/or 3D frames of the subject's teeth 901. The 3D scanner 902 may be configured for transmitting the images and/or 3D frames to a computer system 904. The data transmission may be performed wirelessly or using a cable, such as an ethernet cable, USB cable, or similar. The computer system 904 may be configured for generating a digital 3D model of the scanned object, such as the teeth, and it may further be configured for displaying the 3D model on a display.

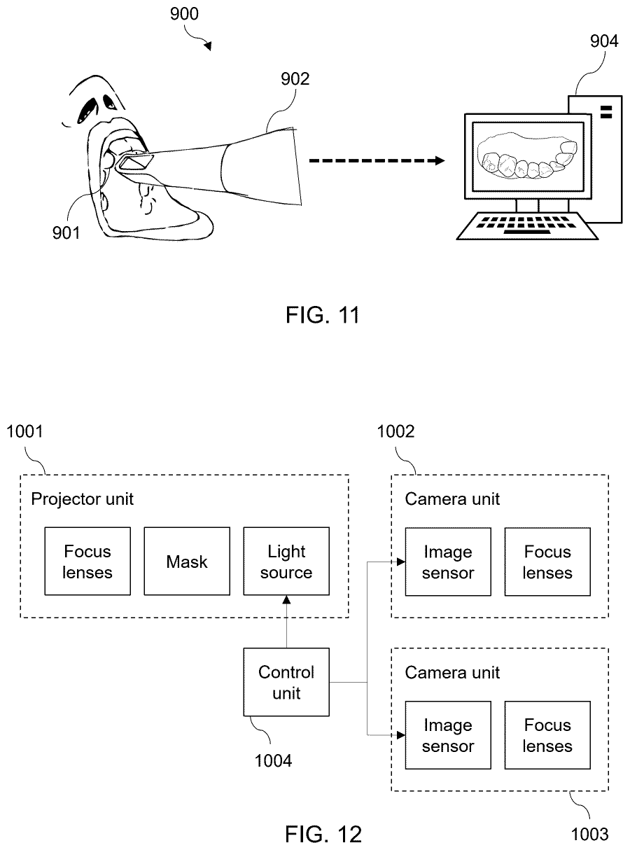

FIG. 12 shows a schematic of some components of an intraoral 3D scanner according to an embodiment of the present disclosure. In this embodiment, the 3D scanner comprises a projector unit 1001, said projector unit comprising a light source, a mask, and one or more focus lenses. The 3D scanner further comprises two or more camera units (1002, 1003), each camera unit comprising an image sensor and one or more focus lenses. In some embodiments, similar optical components are utilized for the focus lenses of the projector unit and the camera units. The 3D scanner further comprises one or more control units 1004 (here exemplified as one control unit) for controlling the operation of the light source and/or the image sensors. As an example, the control unit may be a field-programmable gate array (FPGA) configured for providing a trigger signal to the image sensors for initiating an exposure and/or for initiating the readout of a given image frame. Thus, the image sensors may be synchronized to acquire images simultaneously to generate a set of images from which 3D information can be inferred. The control unit may further be configured for providing a signal to the projector unit for switching the light source on/off at a predetermined time duration and/or frequency. In particular, the control unit(s) may be configured for sequentially switching the light source on and off at a predefined frequency, wherein the light source is turned on for a predefined duration during which all rows of the image sensors are exposed simultaneously. This has the advantage of minimizing motion blur in the acquired images.

### FURTHER DETAILS OF THE INVENTION

1. An intraoral 3D scanner comprising:
at least one projector unit comprising:
   a light source configured for emitting light;
one or more camera units, operatively coupled to the projector unit, each camera unit comprising:
   an image sensor for capturing one or more image frames.
2. The intraoral 3D scanner according to item 1, a pattern generating element configured for generating a pattern of light to be projected onto a surface of an object.
3. The intraoral 3D scanner according to any of the preceding items, wherein the pattern of light is selected from the group of: triangular patterns, hexagonal patterns, square patterns, dot patterns, stripe patterns, fringe patterns, or checkerboard patterns.
4. The intraoral 3D scanner according to any of the preceding items, wherein the image sensor comprises a two-dimensional array of pixels arranged in rows and columns.
5. The intraoral 3D scanner according to item 4, wherein the image sensor is configured for capturing a given image frame by sequentially exposing individual rows within the two-dimensional array of pixels.
6. The intraoral 3D scanner according to any of the items 4 or 5, wherein each row is exposed for a predefined exposure time
7. The intraoral 3D scanner according to any of the preceding items, wherein the 3D scanner further comprises one or more control units configured for sequentially switching the light source on and off at a predefined frequency.
8. The intraoral 3D scanner according to item 7, wherein the light source is turned on for a predefined duration during which all rows are exposed simultaneously.
9. The intraoral 3D scanner according to any of the preceding items, wherein the image sensor is an active-pixel sensor, such as a complementary metal-oxide-semiconductor (CMOS) image sensor.
10. The intraoral 3D scanner according to any of the items 8-9, wherein the predefined duration is between 0.1 ms and 15 ms.
11. The intraoral 3D scanner according to any of the items 8-10, wherein the predefined duration is between 2 ms and 10 ms.
12. The intraoral 3D scanner according to any of the items 7-11, wherein the predefined frequency is between 10 Hz and 100 Hz.
13. The intraoral 3D scanner according to any of the items 7-12, wherein the predefined frequency is between 25 Hz and 40 Hz.
14. The intraoral 3D scanner according to any of the preceding items, wherein the image sensor is configured for reading out a given image frame during a predefined frame readout time.
15. The intraoral 3D scanner according to any of the preceding items, wherein the image sensor comprises a pin for receiving a trigger signal, wherein said trigger signal is configured for initiating the exposure or read out of a given image frame.
16. The intraoral 3D scanner according to item 15, wherein the exposure of each image frame is initiated by the trigger signal.
17. The intraoral 3D scanner according to any of the items 15-16, wherein the control unit(s) are configured for providing the trigger signal to the image sensor(s).
18. The intraoral 3D scanner according to any of the preceding items, wherein the control unit is a device selected from the group of: field-programmable gate array (FPGA), Digital Signal Processor (DSP), Application-Specific Integrated Circuit (ASIC), Microcontroller (MCU), Programmable System-on-Chip (PSoC), or System-on-Chip (SoC).
19. The intraoral 3D scanner according to any of the preceding items, wherein the intraoral 3D scanner comprises two or more camera units, wherein the camera units are synchronized such that they capture images approximately simultaneously.
20. The intraoral 3D scanner according to any of the preceding items, wherein the intraoral 3D scanner comprises four or more camera units, wherein the camera units are synchronized such that they capture images approximately simultaneously.
21. The intraoral 3D scanner according to any of the items 19-20, wherein the camera units are configured for capturing a set of images, wherein each camera unit contributes at least one image to the set of images.
22. The intraoral 3D scanner according to item 21, wherein the number of images in the set of images corresponds to the number of camera units.
23. The intraoral 3D scanner according to any of the items 19-22, wherein the camera units view approximately the same scene or object.
24. The intraoral 3D scanner according to any of the items 19-23, wherein a first image sensor comprised in a first camera unit is configured as the control unit, such that the first image sensor is configured for providing, to each of the remaining image sensors, a trigger signal for initiating the capture of a given image frame.
25. The intraoral 3D scanner according to any of the preceding items, wherein the image sensor is configured with a global reset option where the exposure of all pixel rows starts synchronously.
26. The intraoral 3D scanner according to any of the preceding items, wherein the pattern generating element is selected from the group of: masks such as chrome-on-glass masks, micro mirror arrays (DLP), diffractive optical elements (DOE), or liquid crystal on silicon (LCOS).
27. A 3D scanner system comprising:
the intraoral 3D scanner according to any of the items 1-26; and
one or more processors operatively coupled to the intraoral 3D scanner, wherein the processor(s) are configured for generating a three-dimensional representation of an object based on the captured images.
28. The 3D scanner system according to item 27, wherein the scanner system further comprises a display configured for displaying the three-dimensional representation of the object.
29. A method of generating a three-dimensional (3D) representation of an object, the method comprising the steps of:
sequentially switching a light source on and off at a predefined frequency, wherein the light source is turned on for a predefined duration; and
capturing two or more image frames, wherein each image frame is captured by sequentially exposing individual rows within a two-dimensional array of pixels on an image sensor, wherein each row is exposed for a predefined exposure time, wherein the predefined duration of the light source overlaps the exposure time, such that the object is illuminated in a time period during which all rows are exposed simultaneously.
30. A method of acquiring one or more image frames using an intraoral 3D scanner, the method comprising the steps of:
providing a trigger signal to one or more image sensors forming part of the intraoral 3D scanner, wherein the trigger signal is configured for initiating the exposure or read out of a given image frame;
sequentially exposing individual rows within a two-dimensional array of pixels on each image sensor, wherein each row is exposed for a predefined exposure time, wherein the exposure of a first row in the array is initiated upon receiving the trigger signal;
sequentially switching a light source on and off at a predefined frequency, wherein the light source is turned on for a predefined duration during which all rows in the array are exposed simultaneously; and
reading out a given image frame during a predefined frame readout time, during which the light source is off.
31. The method according to item 30, wherein the intraoral 3D scanner comprises two or more image sensors, and wherein the trigger signal is provided to both image sensors approximately simultaneously.

Although some embodiments have been described and shown in detail, the disclosure is not restricted to such details, but may also be embodied in other ways within the scope of the subject matter defined in the following claims. In particular, it is to be understood that other embodiments may be utilized, and structural and functional modifications may be made without departing from the scope of the present disclosure. Furthermore, the skilled person would find it apparent that unless an embodiment is specifically presented only as an alternative, different disclosed embodiments may be combined to achieve a specific implementation and such specific implementation is within the scope of the disclosure.

1. An intraoral 3D scanner comprising:
at least one projector unit comprising:
a light source configured for emitting light;
a pattern generating element configured for generating a pattern of light to be projected onto a surface of an object;
one or more camera units, operatively coupled to the projector unit, each camera unit comprising:
an image sensor for capturing one or more image frames, wherein the image sensor comprises a two-dimensional array of pixels arranged in rows and columns, wherein the image sensor is configured for capturing a given image frame by sequentially exposing individual rows within the two-dimensional array of pixels, wherein each row is exposed for a predefined exposure time; and
one or more control units configured for sequentially switching the light source on and off at a predefined frequency and for reading out a given image frame during a predefined frame readout time, during which the light source is off, wherein the light source is turned on for a predefined duration during which all rows are exposed simultaneously.

2. The intraoral 3D scanner according to claim 1, wherein the predefined duration is between 0.1 ms and 15 ms.

3. The intraoral 3D scanner according to claim 1, wherein the predefined frequency is between 10 Hz and 100 Hz.

4. The intraoral 3D scanner according to claim 1, wherein the image sensor comprises a pin for receiving a trigger signal, wherein said trigger signal is configured for initiating the exposure or read out of a given image frame, wherein the exposure of each image frame is initiated by the trigger signal.

5. The intraoral 3D scanner according to claim 4, wherein the one or more control units are configured for providing the trigger signal to the image sensor(s).

6. The intraoral 3D scanner according to claim 1, wherein the intraoral 3D scanner comprises two or more camera units, wherein the camera units are synchronized such that they capture images approximately simultaneously.

7. The intraoral 3D scanner according to claim 1, wherein the camera units are configured for capturing a set of images, wherein each camera unit contributes at least one image to the set of images, wherein the camera units have overlapping fields of view, such that the camera units view approximately the same scene or object.

8. The intraoral 3D scanner according to claim 1, wherein a first image sensor comprised in a first camera unit is configured as the control unit, such that the first image sensor is configured for providing, to each of the remaining image sensors, a trigger signal for initiating the exposure or read out of a given image frame.

9. The intraoral 3D scanner according to claim 1, wherein the image sensor is configured with a global reset option where the exposure of all pixel rows starts synchronously.

10. A 3D scanner system comprising:
the intraoral 3D scanner according to claim 1; and
one or more processors operatively coupled to the intraoral 3D scanner, wherein the processor(s) are configured for generating a three-dimensional representation of an object based on the captured images.
